(19) United States
(12) Patent Application Publication
(10) Pub. No.: US 2006/0003316 A1
(43) Pub. Date: Jan. 5, 2006

(54) Immunogenic compositions derived from poxviruses and methods of using same

(76) Inventors: John Simard, Vancouver (CA); John R. Simms, Woodland Hills, CA (US); Zhiyong Qiu, Los Angeles, CA (US)

Correspondence Address: PRESTON GATES & ELLIS LLP, 1900 MAIN STREET, SUITE 600, IRVINE, CA, 92614-7319, US

(21) Appl. No.: 10/620,787

(22) Filed: Jul. 15, 2003

Related U.S. Application Data
(60) Provisional application No. 60/396,293, filed on Jul. 15, 2002.

(51) Int. Cl. C12Q 1/70; C07H 21/02; C12N 15/863; C07K 14/07; A61K 39/12

(52) U.S. Cl. 435/5; 435/69.3; 435/235.1; 435/456; 435/325; 424/186.1; 530/350; 536/23.72

(57) ABSTRACT

Immunogenic compostions composed of poxvirus immunogens and related methods are disclosed. Specifically, immunogenic compostions useful in eliciting immune responses in animals are disclosed. In one embodiment the immunogenic compostions include viral antigens derived from vaccinia and/or variola that elicit cross-reactive immune responses. The immunogens can be made synthetically, by using recombinant DNA technology or derived from purified virus. Moreover, methods of using the immunogenic compostions are also disclosed.

### RELATED APPLICATIONS

This application claims priority from U.S. Provisional Patent Application Ser. No. 60/396,293 filed Jul. 15, 2002, the contents of which are herein incorporated by reference.

### FIELD OF THE INVENTION

The present invention relates to immunogenic compositions and methods for using same. Specifically, the present invention relates to immunogenic compostions prepared from poxvirus-derived immunogens. More specifically the present invention is drawn to immunogenic compostions comprising poxvirus polyproteins that elicit immune responses in animals.

### BACKGROUND OF THE INVENTION

One of the greatest achievements in public health of the last century was the eradication of smallpox. While now absent form the natural environment, variola virus, the causative agent of smallpox, still exists in two designated laboratories, one each in the U.S. and the former U.S.S.R. Additionally it is feared that unacknowledged stocks have been retained in laboratories around the world has a basis for possible biological weapons.

Eradication of smallpox was accomplished through use of a live virus vaccine composed of vaccinia virus, that is cowpox virus. Vaccinia, used by inoculation as a prophylaxis against smallpox, as introduced by Edward Jenner in 1796, is credited as the first vaccine; indeed this is the origin of the terms vaccine and vaccination. Vaccinia's long history and the devastation wrought by smallpox led to acceptance of a high incidence of severe and potentially fatal side effects, even by the standards of half a century ago, despite improved vaccine strains introduced over time.

The prospect of bioterrorism has led to renewed interest in smallpox vaccines. Despite this, most experts today would not endorse widespread use of traditional vaccine strains absent an immediate threat. The frequency and severity of side effects are greater than would generally be considered acceptable today. Additionally, the percentage of the populace that is immune-compromised is much greater now than when vaccinia was in widespread use due to factors such as AIDS and organ transplantation, greatly increasing the potential for debilitating and fatal accidents. More attenuated vaccinia strains developed for use as vaccine vectors have been introduced in recent years, but their effectiveness at inducing protection against smallpox is unknown and, in some views, questionable. Moreover, it is likely that a strain that grew robustly enough to induce protective immunity would still constitute a threat to the immune-compromised.

A non-replicating smallpox vaccine is therefore desirable.

### SUMMARY OF THE INVENTION

The present invention is an immunogenic composition comprising immunogens derived from poxviruses. Specifically, the present invention utilizes poxvirus immunogens to elicit immune responses in animals immunized with immunogenic compostions made therefrom. The traditional smallpox vaccine is composed of viable vaccinia virus. The immunogenic compostions of the present invention utilize carefully selected individual viral proteins, or portions thereof, to elicit cross-reactive immune responses in animals. When made in accordance with the teachings of the present invention these immunogenic compostions induce immune responses that react with closely related poxviruses including vaccinia, variola major and variola minor. Therefore, the present invention provides immunogenic compostions that are safer that the traditional viable vaccinia virus-based vaccines.

In one embodiment of the present invention immunogenic compositions are provided that comprise poxvirus immunogens derived from proteins naturally expressed on the mature virion's surface. In another embodiment of the present invention the poxvirus immunogens comprise immunogenic fragments of the naturally expressed surface proteins. The immunogenic compositions of the present invention can be single proteins, multiple proteins, polyproteins and fragments thereof. The pox virus immunogens can be natural, synthetic peptide, recombinant proteins and/or mixtures thereof. In one embodiment of the present invention the pox virus immunogens are polyproteins incorporating external immunogens of variola or vaccinia surface proteins, nucleic acids encoding such polyproteins, and eukaryotic cells expressing such polyproteins. The poxvirus polyproteins of the present invention can comprise from two or more of the following proteins, or portions thereof: M1R, A36R, I5R, B7R, F8L, and A30L according to the variola major standard nomenclature, or their vaccinia homologues L1R, A33R, H5R, B5R, D8L, and A27L, respectively.

### BRIEF DESCRIPTION OF THE FIGURES

FIG. 1 graphically depicts the immune response of C57/B6 mice immunized with vaccinia virus as measured using a semi-quantitative ELISA procedure.

FIG. 2 graphically depicts the effects of passive transfer of anti-vaccinia immune serum on the survival of C57BL/6 SCID mice following a lethal dose challenge. Panel A depicts the effects of a single administration of various dilutions of the hyperimmune serum. Panel B depicts the effects using weekly administrations of serum.

FIGS. 3-8 depict amino acid alignments between vaccinia and variola surface proteins. For each alignment, the top two entries represent the sequence for vaccinia proteins while the bottom two (or four in FIG. 7) are sequences for variola counterparts. The Genbank accession numbers are indicated in each entry. The vaccinia sequences are from either WR strain (top line) or Copenhagen vaccine strain (second line).

FIG. 9 schematically represents an LAA polyprotein construct in accordance with the teachings of the present invention.

FIG. 10 graphically depicts the effects of passive transfer of anti-LAA polyprotein immune serum on the survival of C57BL/6 SCID mice following a lethal dose challenge.

### DEFINITION OF TERMS

Before proceeding with the detailed description for the present invention it may be beneficial for the reader referred to the following definitions of terms used throughout the remainder of this specification

An “affinity tag” as used herein is a heterologous nucleic acid or amino acid added synthetically to a nucleic acid or peptide to facilitate isolation and purification. For example, and not intended as a limitation, the “affinity tag” may be a series of histidine residues added to a protein or polypeptide, or a poly-adenosine sequence added to a nucleic acid.

A “cocktail of immunogens” is defined herein as a mixture of different immunogens used to form an immunogenic composition.

A “complex of polypeptides” as used herein is any collection of polypeptides used to comprise an immunogenic composition. The polypeptides may be complexed covalently as in the case of a polyprotein, or a protein aggregate wherein the individual proteins are associated with others in the complex via non-covalent intermolecular interactions such as ionic bonds, van der Waal forces, and other forms of protein-protein interactions.

A “cross-reactive” composition is a composition that reacts with complementary compositions and non-complementary compositions. For example, a “cross-reactive immunogen” is an immunogen that elects an immune response that will react with the complementary immunogen as well as immune responses derived from other immunogens. Specifically, as used here in an “immunologically cross-reactive poxvirus” includes a first poxvirus having immunogens that will elicit an immune repose reactive with a second (or more) poxvirus. In one specific, non-limiting example from the present case, immunogens derived from vaccinia virus are used elicit an immune response reactive with variola major and or variola minor viruses.

A “detectable immune response” is any immune response in a mammal that can be quantified, and or identified, using techniques known to those skilled in the art. Such techniques include, but are not limited to immunoassays including enzyme-linked immunosorbant assays, enzyme immunoassays, radioimmune immunoassays, complement fixation, hemeagglutination inhibition, immune precipitation, immunofluorescent assays, in vitro neutralization, T-cell proliferation assays, and or in vivo challenge testing.

An “external immunogen” as used herein refers to surface immunogens that are generally unhindered by obscuring structures or compositions. For example, the external immunogens of the present invention are viral antigens expressed on the surface of the virus particle (virion) and thus exposed to a host's immune system unhindered.

An “external epitope” is a portion of an external immunogen that itself is capable of eliciting, or reacting with an immune response. Note, for the purposes of the present invention a composition need not necessarily be capable of eliciting an immune response in order to react with an immune response and visa versa.

An “immunogen” is any material that, by itself, or in combination with other compounds, elicits a detectable immune response. Specifically, as used herein an immunogen is a viral protein or polyprotein. More specifically, an immunogen is a protein, lipid, lipoprotein protein, or polyprotein derived from a poxvirus. “Immunogen” as used herein includes external immunogens and external epitopes as defined herein. In other embodiments of the present invention an “immunogen” can include a DNA expression cassette containing one or more viral nucleic acid sequence encoding for proteins, lipids, lipoproteins or polyproteins. Moreover, the viral proteins, lipids, lipoproteins or polyproteins of the present invention can be extracted from a natural virus, produced recombinantly ex vivo, produced recombinantly in vivo, or chemically synthesized. Furthermore, any and all possible combinations of “immunogens” are envisioned.

An “immunogenic composition” is defined herein as a preparation that when administered to a mammal elicits an immune response. The mammal's immune response can be humoral, cellular or any combination thereof. The immunogenic compositions can comprise a single immunogen, multiple immunogens or nucleic acid encoding for the immunogen. The immunogenic composition can also comprise one or more adjuvants, carriers, emulsifying agents, solvents, or other excipient known to those having ordinary skill in the art of immunology. The immunogenic compositions of the present invention can be administered by any technique know those skilled in the art including, but not limited to intramuscular injection (IM), intradermal injection (ID), intracranial injection (IC), intraperitoneal (IP), intranodally (IL), trasdermally, orally, intravaginally, rectally, occularlly and combinations thereof.

An “isolated nucleic acid” is a nucleic acid the structure of which is not identical to that of any naturally occurring nucleic acid or to that of any fragment of a naturally occurring genomic nucleic acid spanning more than three separate genes. The term therefore covers, for example, (a) a DNA which has the sequence of part of a naturally occurring genomic DNA molecules but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Specifically excluded from this definition are nucleic acids present in mixtures of (i) DNA molecules, (ii) transfected cells, and (iii) cell clones, e.g., as these occur in a DNA library such a cDNA or genomic DNA library.

A “neutralizing antibody” as used herein is any antibody that is capable of preventing viral replication. Specifically, a neutralizing antibody is an antibody that reacts with and binds to a viral protein, or a portion therefore, that results in a viral protein-antibody complex. Neutralizing antibodies can work in a variety of ways. For example, and not intended as a limitation, a neutralizing antibody made in accordance with the teachings of the present invention can bind with a variola external immunogen thus preventing host cell recognition and entry. Consequently viral replication is blocked and the infection is neutralized

The “percent identity” of two amino acid sequences or of two nucleic acids is determined using the algorithm of Karlin and Altschul (Proc. Natl. Acad. Sci. US 87:2264-2268, 1990), modified as in Karlin and Altschul (Proc. Natl. Acad. Sci. USA 90:5873-5877, 1993). Such an algorithm is incorporated into the NBLAST and XBLAST programs of Altschul et al. (J. Mol. Biol. 215:403-410, 1990). BLAST nucleotide searches are performed with the NBLAST program, score=100, wordlength=12, to obtain nucleotide sequences homologous to a nucleic acid molecule of the invention. BLAST protein searches are performed with the XBLAST program, score=50, wordlength=3, to obtain amino acid sequences homologous to a reference polypeptide (e.g., SEQ ID NO:2). To obtain gapped alignments for comparison purposes, Gapped BLAST is utilized as described in Altschul et al. (Nucleic Acids Res. 25:3389-3402, 1997). When utilizing BLAST and Gapped BLAST programs, the default parameters of the respective programs (e.g., XBLAST and NBLAST) are used. See http://www.ncbi.nim.nih.gov.

A “polyprotein” is more than one protein, or polypeptide, made as a result of a single transcriptional event that has not been cleaved into individual protein, or polypeptide chains. The polyprotein can, or can not have linker regions connecting the individual proteins or polypeptides. Linker-spaced peptide as used herein will mean a region having at least one amino acid separating two individual proteins or polypeptides wherein the linker amino acid(s) is not considered part of the naturally occuring protein or polypeptide. Moreover, a polyprotein according to the present invention can also be synthesized synthetically wherein individual amino acid residues are chemically combined to make the polyprotein.

A “poxvirus membrane-associated protein” includes, but is not limited to variola proteins M1R, A36R, I5R, B7R, F8L, and A30L; and their vaccina homologs L1R, A33R, H5R, B5R, D8L, and A27L. As used in the claims the Markush group M1R, A36R, I5R, B7R, F8L, A30L, L1R, A33R, H5R, B5R, D8L and A27L refers to these variola and vaccinia proteins.

A “synergistic antibody” as used herein defines a first antibody that enhances the effects of a second antibody. For example, a first antibody can enhance the virus neutralizing effects of a second antibody. The first antibody need not be neutralizing itself in order to act synergistically with the second antibody. Moreover, an antibody can be synergistic to more than one antibody (to greater, equal, or lesser degrees), or a combination of antibodies can exhibit synergy with one or more other antibodies.

A “subunit vaccine” as used herein refers to an immunogenic composition comprising specific portions of identified immunogens, but not the entire virus itself. For example, and not intended as a limitation, a “subunit” includes immunogens, external immunogens and external epitopes as defined herein. In one embodiment of the present invention “subunit vaccines” comprise recombinantly produced immunogens.

A “target antigen,” or “target immunogen” as used herein refers to an immunogen derived from variola minor itself rather than cross-reactive immunogens derived from either vaccinia or variola minor.

The term “topology” as used herein refers to the tertiary structure, or three-dimensional shape of an immunogen. Topology is used herein to describe the immunogens final structure including post transcriptional processing. The topology of an immunogen often defines the binding avidity and affinity exhibited between homologous immunogen-antibody pairs.

A “variola consensus sequence” as used herein refers to a series of related DNA, RNA or protein sequences derived from (or calculated for) Variola. A consensus sequence generally is a sequence that reflects the most common choice of base or amino acid at each position. Areas of particularly good agreement often represent conserved functional domains. Thus, by establishing a variola nucleic acid consensus sequence for a target protein that induces neutralizing or synergistic antibodies a recombinant protein can be made that will be broadly cross-reactive between vaccinia and variola strains.

A “variola homologue” is as used herein is a nucleic acid or peptide sequence derived from variola virus that corresponds with a nearly identical sequence of the same protein in vaccinia. Variola homologue can also be used herein to define a nucleic acid or peptide sequence derived from variola minor virus that corresponds with a nearly identical sequence of the same protein in variola major (and visa versa).

### DETAILED DESCRIPTION OF THE INVENTION

The apparent basis for the effectiveness of vaccinia as a vaccine for smallpox is the ability of antibody elicited by vaccinia proteins to cross-react with homologous proteins from variola. Past attempts to create a safer, killed virus vaccine failed due to a lack of understanding of the life-cycle of the virus and when critical antigens were incorporated into the virion. When smallpox vaccines were last in wide use the prospect of recombinant subunit vaccines had not yet arisen. Herein we disclose immunogenic compositions comprising extraviral regions of poxvirus membrane-associated proteins (herein after referred to collectively as immunogens) that are useful in generating immune responses against vaccinia and variola viruses.

Five vaccinia immunogens, A27L, B5R, D8L, H5R, and L1R, have been identified and associated with the infection process and neutralizing antibodies induction. (Galmiche, M. C. et al., Virol. 254:71-80, 1999; Gordon, J. et al., Virol. 181: 671-686, 1991; Hsiao J-C. et al., J. Virol. 73:8750-8761, 1999; Hooper, J. W. et al., Virol. 266:329-339, 2000; Ichihashi, Y.and Oie, M., Virol. 220:491-4, 1996; Law, M. and Smith, G. L. Virol. 280:132-142, 2001; Rodriguez, J. F. et al., J. Virol. 61:3550-3554, 1987; Wolffe E. J. et al., Virol. 211:53-63, 1995). Antibodies against a sixth protein, A33R, while generally reported to not be neutralizing in its own right, increased the effectiveness of, or synergizes with, other antibodies (Hooper, J. W., et al., Virol. 266:329-339, 2000). Additional proteins involved in virus entry and/or neutralization, including A34R, A36R A56R, F12L, F13L, A14L, and A17L are also immunogenic. (Hsiao J-C. et al., J. Virol. 73:8750-8761, 1999; Law, M. and Smith, G. L. Virol. 280:132-142, 2001).

The aforementioned immunogens exhibit a wide variety of topologies and modes of association including type I and II integral membrane proteins, lipid anchors, and protein-protein interaction. However, it is not necessary that the immunogens used to make the present immunogenic compositions exhibits these features; however, in some embodiments of the present invention these immunogen properties can be advantageous. In naturally mature virions at least part of the immunogen is located on the particle's surface (external immunogen) and therefore accessible to the host's immune response. Therefore, in one embodiment of the present invention the immunogenic compositions are comprised of the entire external immunogen. In another embodiment of the present invention external epitopes derived from virus external immunogens are used.

With recombinant subunit vaccines there is no danger of reversion to or survival of virulent virus, as there is with attenuated or killed vaccines, respectively. Thus, immunogenic compositions comprising target antigens instead of, or in addition to, cross-reactive antigens can be used without fear of infection. Although variola major genomic DNA is not generally available, the sequence of several strains is readily accessed through the major genetic databases. Variola major homologs of vaccinia proteins are frequently identified in Entrez and Swiss Protein database entries of the vaccinia proteins. A BLASTP search can also quickly identify homologs, as can a variety of other text and sequence searches. Once a homologous protein sequence is identified a sequence for an encoding nucleic acid can be determined by reference to the underlying gene sequence in the database, or by conceptual back-translation from the protein sequence. In the event that the immunogen will be produced in a non-mammalian host the latter technique offers an opportunity to optimize codon usage. Encoding nucleic acid can be physically created with routine techniques including, gene synthesis by assembly of synthetic oligonucleotides and PCR using vaccinia DNA as a template and mutagenic primers. RNA can be obtained in a secondary step using, for example, the T7 in vitro transcription technology.

The same procedure can also be carried out with variola minor sequences. Variola minor caused a less severe form of smallpox and thus is less likely to be weaponized. However, variola minor nucleic acid and amino acid sequences are generally more similar to variola major than the corresponding vaccinia sequences. Therefore, immunogenic compositions comprising variola minor immunogens are also considered within the scope of the present invention.

The immunogenic compositions of the invention can take on several embodiments. The composition can be a mixture of immunogens including, external immunogens or external epitopes, or one or more polyproteins, that is several proteins, or portions thereof, produced as a single translation product. The composition can also be a nucleic acid molecule, or molecules, encoding the above polypeptides, administered as a nucleic acid vaccine as described, for example, in U.S. Pat. No. 6,214,804 entitled “INDUCTION OF A PROTECTIVE IMMUNE RESPONSE IN A MAMMAL BY INJECTING A DNA SEQUENCE”. In further embodiments the immunogenic composition can be a complex of proteins or protein segments. In one such embodiment the individual polypeptide sequences are modified by the addition of biotinylation sites. Following biotinylation, multimeric complexes can be formed by reacting the polypeptides with avidin or streptavidin. In another such embodiment the membrane anchors naturally present in the proteins are retained, or added to such proteins lacking them, and the proteins are incorporated into lipid vesicles or micelles. Any such complex can include a single or multiple polypeptide species. Single species complexes, which could themselves be combined in a cocktail so that the immunogenic composition contained multiple target antigens. In this embodiment the immunogen's multivalent nature can reduce or eliminate the need for an adjuvant(s).

Purified subunit vaccines, whether comprised of naturally occurring or recombinantly produced polypeptides are generally inherently poorly immunogenic. To improve their immunogenicity they can be formulated with an adjuvant. Currently, there is only one FDA-approved adjuvant for human use available, aluminum hydroxide or alum (one commercially available product is BioVant™, BioSante Pharmaceuticals). Although alum is the only U.S. approved adjuvant for general human use in a variety of vaccines and immunizations, alternative preparations can be licensed in other countries, for example incomplete Freund's adjuvant (IFA) or MF59.

The immunogenic compostions of the present invention are useful in eliciting immune responses in animals against vaccinia, variola major, variola minor and other, related poxviruses. The immunogenic compostions of the present invention can be formulated with or without adjuvant compostions. Briefly, the immunogens of the present invention can be purified from the cellular milieu using methods known to those having ordinary skill of the art in protein chemistry. Techniques such as, but not limited to, anion exchange chromatography, gel filtration, affinity chromatography, gel electrophoresis and/or density gradient purification can be used either alone, or in combination with other techniques to isolate and purify the immunogens of the present invention. Once purified the immunogens of the present invention are resuspended in a pharmaceutically acceptable buffer and then mixed with pharmaceutical excipients as needed to maintain the immunogen's stability, topology, and potency (collectively referred to as “immunogenicity”).

Persons having ordinary skill in the art would only need routine experimentation in order to ascertain the final immunogen composition, routes of administration and dosages to achieve their intended results. For example, and not intended as a limitation, if a immunoprophylactic composition was desired, the selected immunogen(s) could be compounded in simple pharmaceutical buffers and administered with or without an adjuvant. Different concentrations would be prepared ranging from approximately 0.001 mg/mL to 500 mg/ml or more depending on immunogen solubility. Next, a measured amount of the composition would be administered to the animal based on body weight and the resulting immune response for each dose would be determined (see Example 5 below). If the initial immune responses fell below desired levels, boosters could be administered following the same protocol as previously described.

Routes of administrations can include, but are not limited to intramuscular injection (IM), intradermal injection (ID), intracranial injection (IC), intraperitoneal (IP), intranodally (IL), trasdermally, orally, intravaginally, rectally, occularlly and combinations thereof. Efficacy of the immunoprophylactic composition would then be established by challenging the animal with a lethal or sub-lethal dose of virus as described in Example 6 below. An immunogenic composition made in accordance with the teachings of the present invention would be considered immunoprophylactic if the animals receiving the immunogenic composition had a higher survival rate than those receiving a placebo composition.

### EXAMPLES

The invention is illustrated by the following Examples. These Examples are presented for illustrative purposes only and are not intended to limit the invention.

### Example 1

### Antibodies Provide Protection from Vaccinia Infection

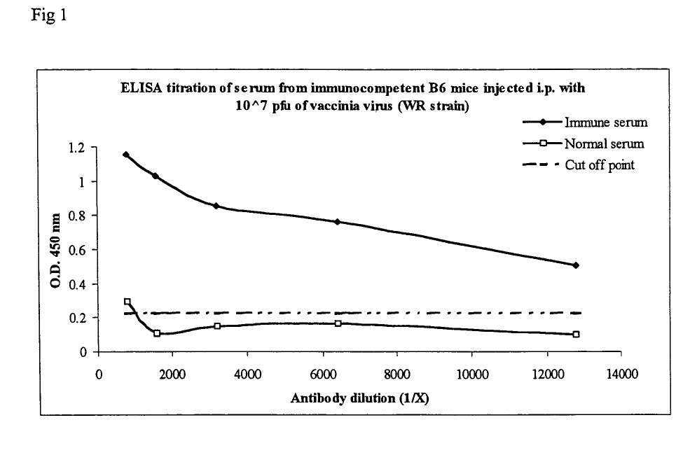

To verify that protection against poxvirus infection is antibody mediated adoptive transfer experiments into SCID (severe combined immunodeficiency) mice exposed to vaccinia virus were carried out. SCID mice lack both B and T cells and are incapable of mounting an antibody or T cell response of their own against a vaccinia virus challenge. Thus any protective effect observed would be due to the ability of the adoptively transferred serum to neutralize the inoculated virus. C57BL/6 (B6) mice are immunocompetent and able to eliminate low level vaccinia infections. Therefore to generate antisera for adoptive transfer, we infected 10 C57BL/6 mice with vaccinia virus and 14 days later bled the animals to obtain a high titer antiserum against vaccinia virus (see FIG. 1). Specifically, 1 μl of trypsin (25 mg/ml) was added to each of six 100 μl aliquots of WR strain vaccinia virus (2.5×10⁸ pfu/ml) and incubated in a 37° C. waterbath for 30 minutes. 399 μl of PBS was then added to each aliquot each of which were vortexed and placed on ice. Ten female B6 were injected IP (intraperitoneally) with 200 μl of this virus solution (10⁷ pfu). On day 14 following infection five surviving animals were sedated and bled from the retro-orbital plexus yielding approximately 2 ml of pooled immune serum.

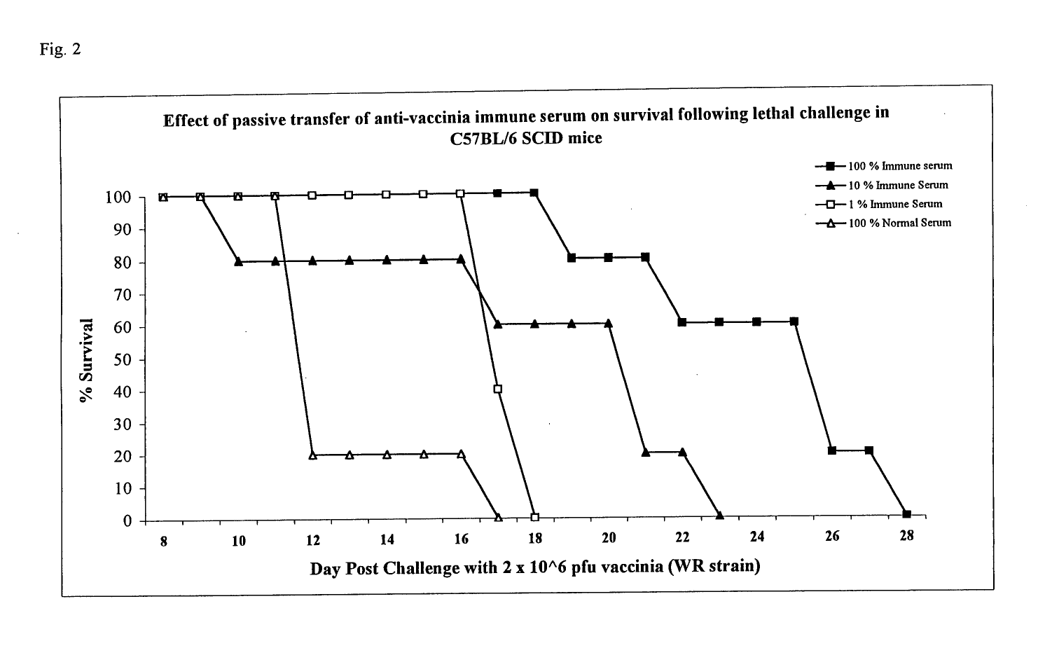

Four groups of five SCID mice each were injected IP with 200 μl of neat, 1:10, or 1:100 diluted, immune serum or 200 μl of normal mouse serum, respectively. After allowing three days for the serum to redistribute the mice were infected with 2×10⁶ pfu of vaccinia (similarly prepared as above). This inoculum would be expected to produce mortality in approximately 9 days in unmanipulated SCID mice (Selin et al., J. Immunol. 166:6784-6794, 2001). The results from this experiment (see FIG. 2A) showed that mice receiving undiluted antiserum survived longer than mice administered non-immune serum derived form uninfected C57BL/6 animals. The protective effect also titrated, as evidenced by intermediate protection of SCID mice receiving diluted anti-vaccinia antibodies. Mice receiving antibodies were not fully protected as they eventually succumbed to the virus, however, these animals were incapable of producing additional antibodies in response to the virus infection and received no supplemental administration of serum after the initial dose. If supplemental administrations of hyperimmune serum are given protection can be prolonged (see FIG. 2B).

### Example 2

### Alignment of Vaccinia Neutralization Targets with Varioloa Major Homologs

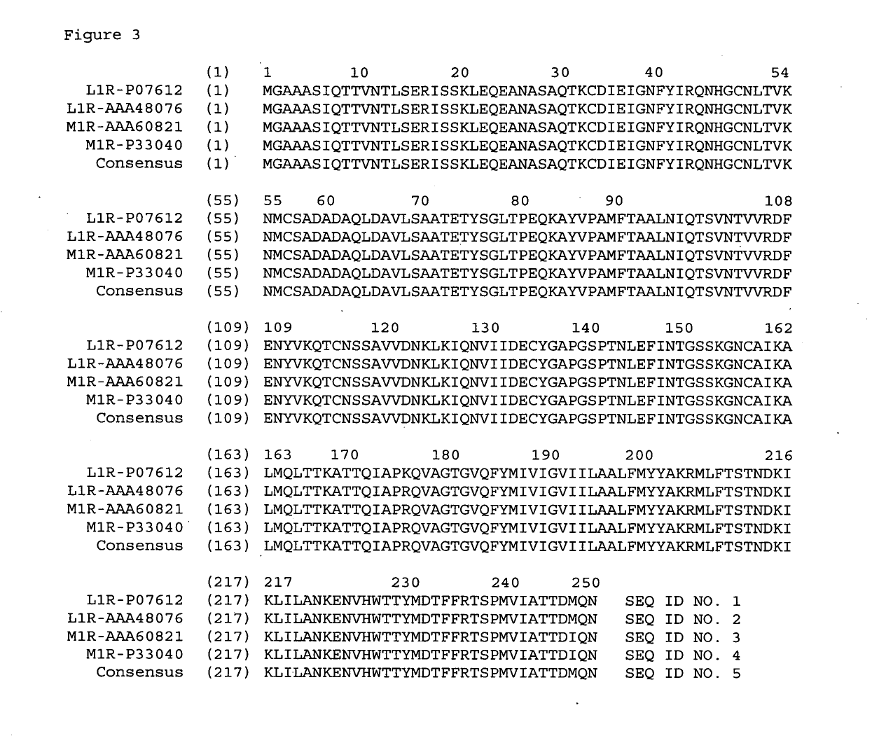

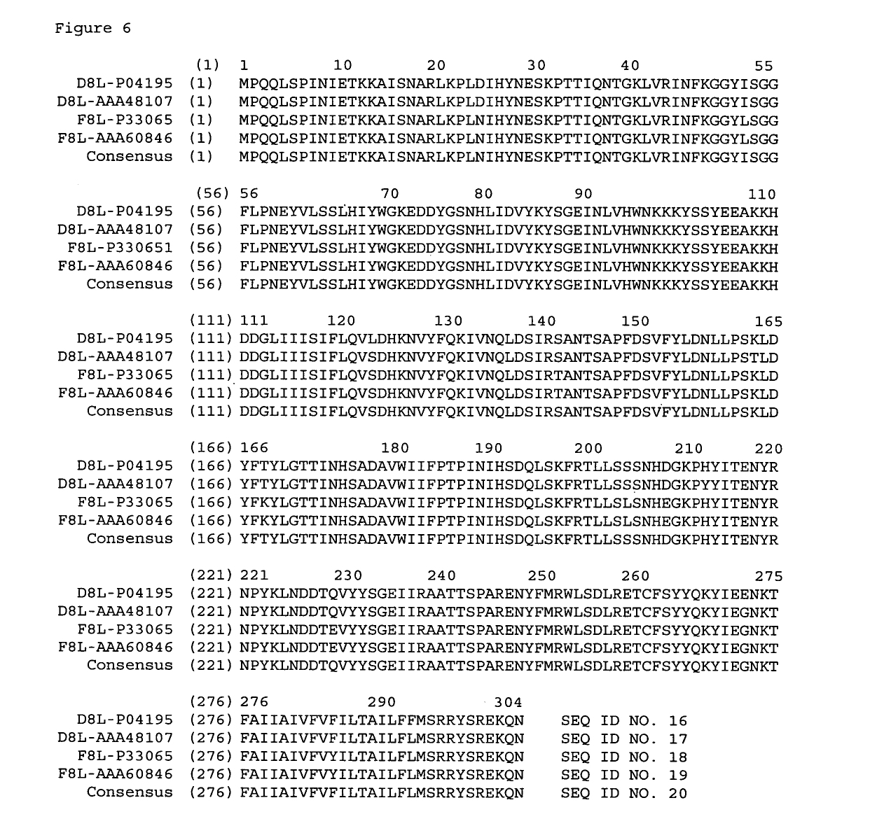

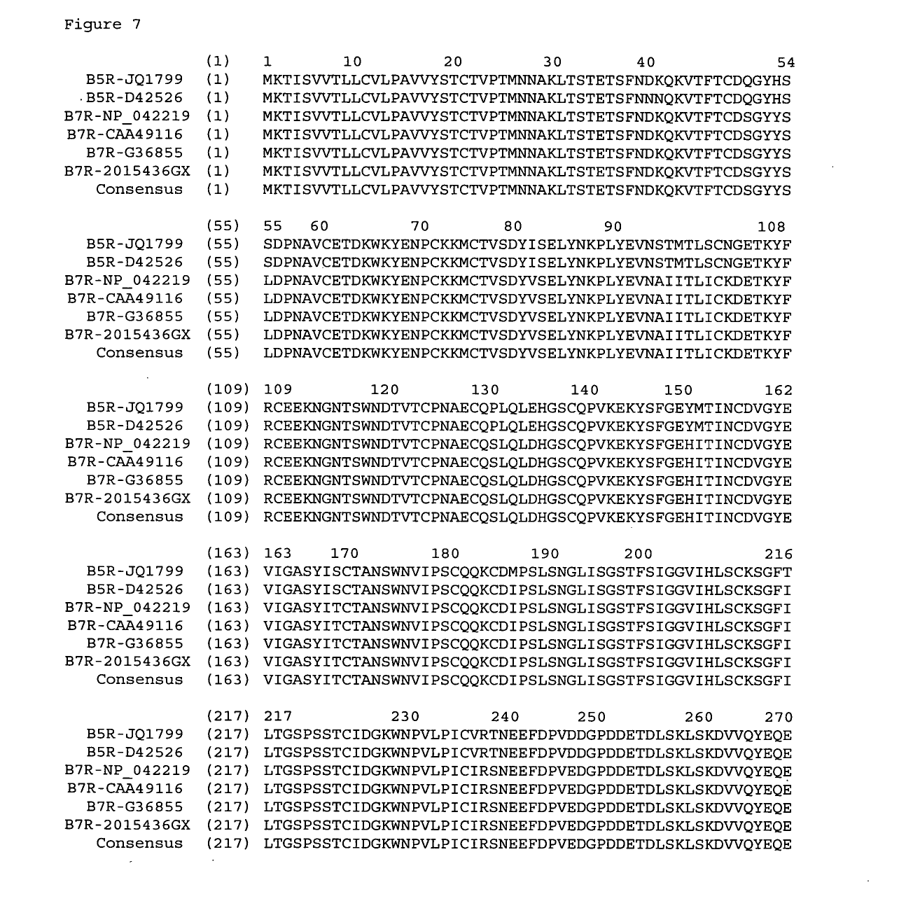

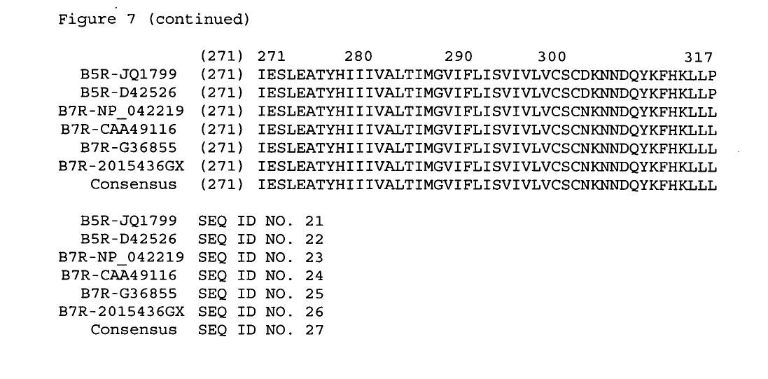

The Entrez Protein databank has multiple entries for each of the target antigens of both viruses. By aligning these sequences differences between vaccinia and variola are readily observed (see FIGS. 3-8). Some pairs are nearly identical. (In the following pairings the vaccinia protein or amino acid residue will always precede the variola major counterpart). For L1R-M1R there are only two positions out of 250 that showed any variation, conservative K-R and M-I substitutions, and the K is reported in only one of the two vaccinia sequences used. Other pairs showed more substantial variation. A27L-A30L showed variation at 7 positions out of 110 though in four cases only one of the four sequences used differed. Moreover some of the substitutions were decidedly non-conservative, for example the R-A substitution at position 30. In a similar manner D8L-F8L varied at 15 of 304 positions. A33R-A36R varied at 11 of 185 positions. In this case, both vaccinia and both variola sequences were identical to each other, the variola sequences had a deletion, at position 73, relative to vaccinia. Still other pairings showed greater differences. B5R-B7R varied at 24 of 317 positions with greater clustering of substitutions than in the above pairings. For H5R-15R it is difficult to simply state the degree of variation. There are at least 9 simple substitutions in 221 positions. Additionally there are at least three deletions in vaccinia relative to variola, though different alignments in these regions, particularly following position 70, can effect the count of substitutions and deletion/insertion events.

### Example 3

### Construction of a Polyprotein-encoding Nucleic Acid

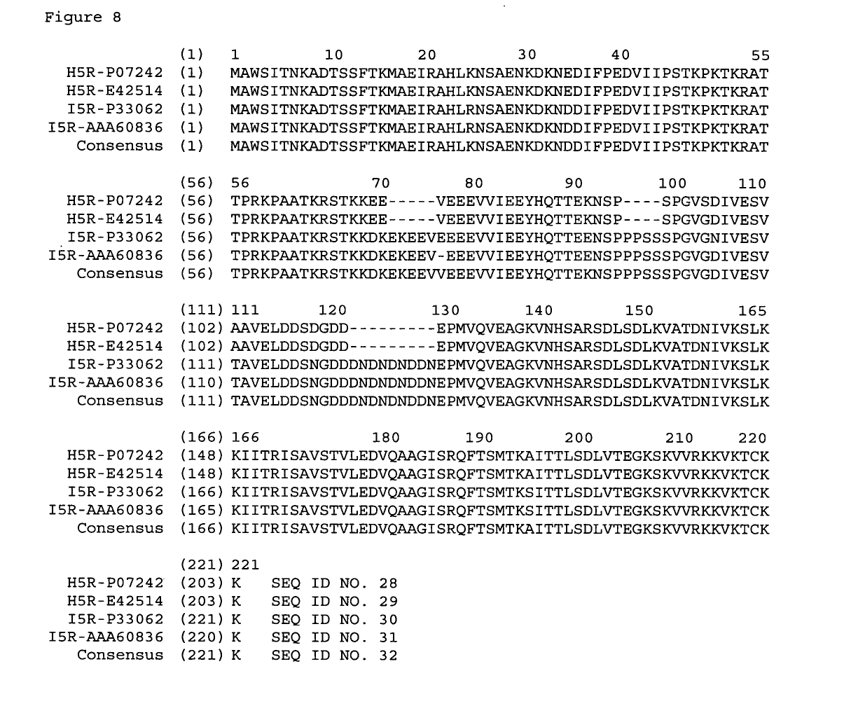

Examination of the sequence of L1R-M1R revealed a likely signal sequence at its N-terminus and hydropathy analysis suggested the existence of a membrane spanning domain beginning around position 187. Taking advantage of its natural signal sequence this protein was chosen as the N-terminal component of the polyprotein, thus insuring transport into the lumen of the endoplasmic reticulum and thereby exposing the polyprotein to glycosylating enzymes. Glycosylation can be important to native antigenicity of membrane proteins. This can also promote secretion into the culture medium, simplifying purification. Because neutralizing antibodies are expected to be directed against the extraviral segment of the protein, and to avoid the protein becoming anchored in the membrane, the amino acids after position 186 were not included in the construct. PCR was carried out using vaccinia strain WR as template. The 5′ primer added and AfIII restriction site and the 3′ primer changed the K at position 176 to the R found in the other sequences (see FIGS. 3 and 9) and added part of a GGGGSSGG spacer-linker sequence following position 186, thereby incorporating a BamHI site near the 3′ end of the amplicon. This product was then cloned into the plasmid expression vector PCDNA3.1©(+) (Invitrogen Corporation, Carlsbad, Calif.) between its AfIII and BamHI sites. This plasmid was prepared by standard means and digested with AfIII and EcoRI in anticipation of the three-fragment ligation described below.

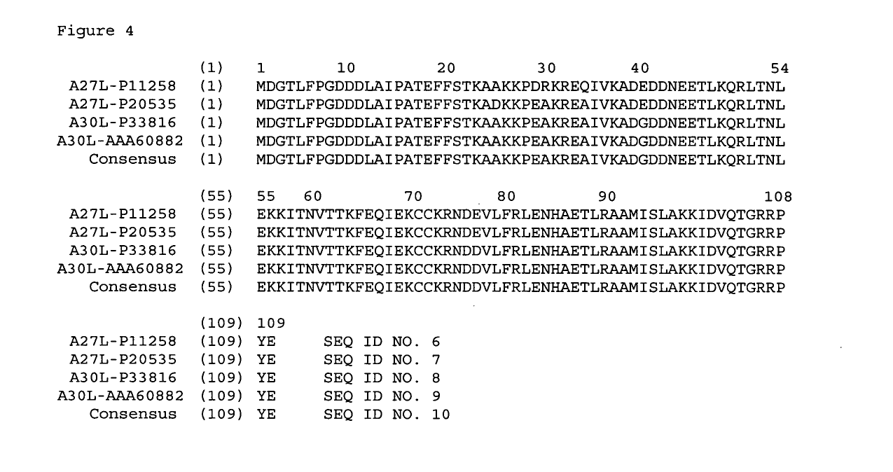

A27L-A30L has no obvious membrane anchor in its sequence and rather may associate with the membrane through interactions with another protein, possibly A17L. Thus the whole protein was included in the construct. Due to its relatively small size and closely grouped substitutions this coding sequence was built from overlapping synthetic oligonucleotides. This product included a BamHI site and the remainder of the GGGGSSGG spacer-linker sequence at its 5″ end. It also incorporated an N found at position 42 as found in variola major strain India-1967, rather then the D found in the other sequences in FIGS. 4 and 9. Its 3′ end added most of a GGGGSSGG spacer-linker sequence that incorporated a BspEII site. Due to low efficiency in the annealing and assembly of this construct it was then amplified to generate ample material for cloning. After digestion with BamHI and BspEII this fragment was used in the three-fragment ligation described below.

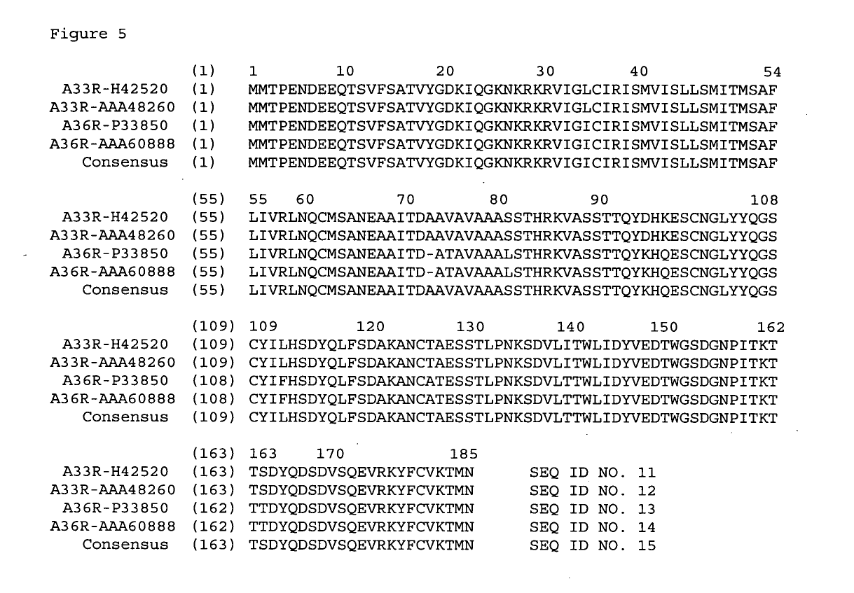

A33R-A36R is a type II membrane protein, that is, the N-terminus is cytoplasmic and the C-terminus is external. A hydrophobic sequence that can function as a membrane anchor end at position 57. Amino acids 57 through the C-terminus were included in the construct. In order to incorporate all the variola-specific residues a series of PCR reactions was carried out using a total of 6 oligonucleotide primers. Initially the needed segment of vaccinia was amplified. The 5′ most primer completed the GGGGSSGG spacer-linker sequence that incorporated a BspEII site at the 3′ end of the A33R-A36R construct. Further rounds of amplification were carried out to generate the internal substitutions. In a final round of amplification the 3′ most primer added a third GGGGSSGG spacer-linker sequence, a 10 histidine tag, and an EcoRI site. Following digestion with BspEII and EcoRI this fragment was combined with the two above in a three-fragment ligation generating an expression vector with a reading frame encoding M1R₁₋₈₆, a GGGGSSGG spacer-linker, A30L, another spacer-linker, A36R₅₇₋₁₈₄, a third spacer-linker, and a histidine tag, under the control of a CMV promoter (see FIGS. 5 and 9). The resultant polyprotein was named LM.

### Example 4

### Polyprotein Expression and Purification

The LAA expression vector plasmid was prepared by standard means and transfected into HEK 293 cells with the aid of a cationic lipid preparation. Cell lysates and culture supernatants were applied to chromatography columns containing Ni-charged resin to capture the LAA polyprotein by its his-tag. The eluate was analyzed for presence of the polyprotein by western blot using an anti-vaccinia antiserum prepared as described in example 1. A single sharp band consistent with the expected molecular weight, ˜68 kD, was detected.

### Example 5

### Anti-Vaccinia Antibodies Cross-react with LAA Protein

To demonstrate cross-reactivity between vaccinia antibodies and LAA protein a competition ELISA was devised. ELISA plate wells were coated with vaccinia virus or elution fractions from a nickel column used to purify the LAA protein from culture supernatant (histag fractions). The wells were reacted with normal or vaccinia-immune sera from B6 mice in the presence or absence of a competitor. For histag fraction coated wells vaccinia virus was used as the competitor. For vaccinia virus coated wells histag fractions were sued as the competitor. More specifically, the wells were coated by overnight incubation with antigen in a buffer of 20 mM sodium borate, pH 9.5, at 2-8° C. the wells were washed twice with water and blocked with an ELISA diluent containing 2% normal goat serum and 5 mg/ml casein in tris-buffered saline. The wells were again washed twice with water and then incubated 3 hr. at room temp. with normal serum, immune serum, or immune serum plus competitor; final dilution of serum 1:100 in ELISA diluent. The plates were then processed as standard with biotinylated goat anti-mouse Ig, streptavidin-alkaline phosphatase, and PNPP substrate, and absorbance read at 405 nm. Competition was observed in both directions (see table below) indicating that anti-vaccinia antibodies can recognize the LAA protein.

| | | | Net** | |
|---|---|---|---|---|
| | | | A405 | Relative |
| Antigen | Serum | Competitor | 60 min. | response |
| Histag Fraction 1 | Immune | none | 0.153 | 100.0 |
| Histag Fraction 1 | Immune | vaccinia virus | 0.011 | 6.9 |
| Histag Fraction 1 | Normal* | none | 1.060 | |
| Histag Fraction 2 | Immune | none | 0.073 | 100.0 |
| Histag Fraction 2 | Immune | vaccinia virus | −0.091 | “0” |
| Histag Fraction 2 | Normal | none | 1.001 | |
| Histag Fraction 3 | Immune | none | 0.081 | 100.0 |
| Histag Fraction 3 | Immune | vaccinia virus | −0.085 | “0” |
| Histag Fraction 3 | Normal | none | 0.948 | |
| Vaccinia Virus | Immune | none | 1.629 | 100.0 |
| Vaccinia Virus | Immune | Histag Fraction 2 | 1.203 | 73.8 |
| Vaccinia Virus | Immune | Histag Fraction 3 | 1.140 | 70.0 |
| Vaccinia Virus | Normal | none | 0.528 | |
| H2O | Immune | none | 0.000 | 0 |

*Pre-immune serum was not available and this normal serum exhibited an unusual reactivity.
**Net A405: Difference in Absorbance at 405 nm. of a well and a water coated well

### Example 6

Use of the Polyprotein as an immunogenGroups of B6 mice are immunized IM with increasing doses (between 5 and 100 μg per dose) of the LAA polyprotein absorbed to aluminium hydroxide (Alhydrogel or BioVant™). Booster inoculations are administered repeatedly until high levels of virus-specific antibodies are obtained as judged by ELISA.

The ability of this antiserum to neutralize vaccinia virus is determined using an in vitro neutralization test. Specifically, a 1:10 dilution of test antiserum in minimal essential medium (MEM) is heat inactivated for 1 hr. in a 56° C. waterbath. A 1:2 dilution series of hyperimmune antiserm is prepared in a 96-well flat-bottomed microtiter plate with 50 μl of diluted serum in each well. Subsequently 50 μl of vaccinia virus (WR strain), prepared at 100 TCID₅₀, is added to each well. (TCID₅₀, for tissue culture infection dose 50, is the amount of virus needed to infect half of the cells in a culture). Plates are incubated at 37° C. for 1 hr. followed by the addition of 100 μl MEM containing 3×10⁵ CV-1 cells/ml to each well. Plates are incubated overnight and finally 20 μl of ALAMAR BLUE™ is added. Following a further overnight incubation the optical density (O.D.) of each well is read at 620 nm using an ELISA plate reader. Each serum is tested in triplicate with appropriate positive (no virus added) and negative (no cells added) controls. The neutralization titer is taken as the highest dilution of test serum that gives a reduction in O.D. of greater than 50% when compared to the net difference between the positive and negative control wells. ALAMAR BLUE™ is a non-toxic redox indicator that allows the innate metabolic activity of cells to be monitored colorometrically. When cells have been killed by lytic infection with vaccinia virus this metabolic activity ceases and the indicator dye remains in its original oxidized state. Any neutralizing antibodies present in the serum will bind to the surface of the virions and prevent the cells from becoming infected by the vaccinia virus. The extent of virus neutralization in each well is directly correlated with the net change in O.D. for that well.

Analogous protocols are carried out in cynomolgus monkeys to demonstrate immunogenicity in primates.

### Example 7

### Anti-LM Antibodies Protect Against Vaccinia Challenge

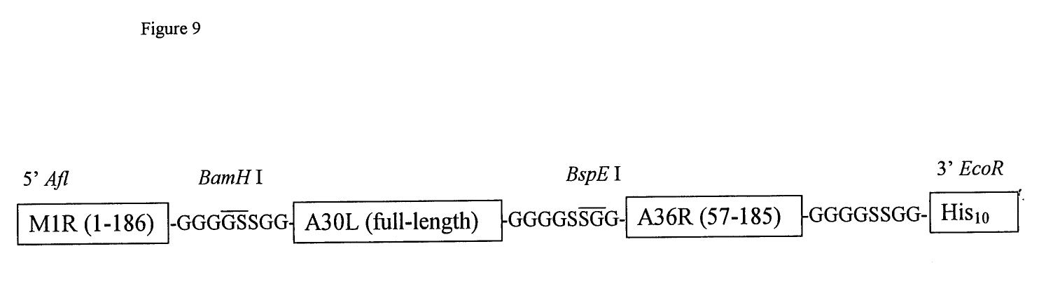

B6 mice were immunized with a single injection of 10 μg of the LAA polyprotein absorbed to aluminium hydroxide (Alhydrogel). The mice were bled 21 days later and immune serum was prepared. Passive transfer was carried out essentially as described in Example 1 above with 12 mice receiving single doses of immune serum and 6 receiving single doses of normal serum. As seen in FIG. 10 all of the mice receiving normal serum have survived by day 20, whereas 60% of the mice receiving immune serum still survived.

### Example 8

### Anti-LAA Immunization Protects Against Vaccinia Infection

B6 mice are immunized with LAA polyprotein absorbed to aluminum hydroxide to obtain a high titered antiserum (as optimized in example 5). Immune serum is then administered (200 μl IP) to SCID mice and allowed to redistribute for 48-72 hr. Mice are then infected with 2×10 pfu of trypsinized vaccinia virus and observed daily for morbidity and mortality. Control animals are administered 200 μl of normal mouse serum in a similar fashion. Protection is indicated by the prolonged survival following virus challenge of those animals receiving the immune serum, as compared to those receiving non-immune serum.

We also demonstrate that repeated administration of immune serum on a weekly basis to SCID mice rescues the animals from an otherwise lethal infection for the duration of the experiment (70 days; Li, J. S. et al., J. Immunol. 166:1855-62, 2001). This demonstrates that maintaining high circulating levels of neutralizing antibodies for prolonged periods of time, as would be the case in an immunocompetent vaccinee, will confer a long lasting protective effect.

B6 mice are immunized with LM polyprotein absorbed to aluminium hydroxide to induce a high titer of neutralizing antibodies as optimized in example 5. Wild type B6 mice are resistant to low level infection with vaccinia virus strain WR, however these animals are susceptible to lethal infection with a high dose of virus (˜10⁸ pfu). By inoculating groups of 5 unimmunized B6 mice IP with an increasing log dilution series of vaccinia virus (10⁵, 10⁶, 10⁷ etc. pfu per dose) the virus is titrated to determine the 50% lethal dose of virus (LD50) as described by Reed, L. J. and Muench, M. (American Journal of Hygiene 27:493-497, 1938). Vaccinated immunocompetent B6 animals and unvaccinated controls are administered a lethal dose (10 LD50) of vaccinia virus strain WR 14 days after IM immunization with LM polyprotein. This type of challenge would be expected to be fatal to unimmunized animals after a 3 to 5 day incubation period (Hooper, J. W. et al., Virol. 266:329-339). A similar experiment conducted in primates (rhesus macaque, cynomolgus monkey) is used as a closer approximation of human disease. Primates are challenged with a strain of vaccinia virus. Dosing studies, similar to those performed in mice (see example 5) are performed to optimize the vaccination schedule.

### Example 9

### Determination of the Extent of Protection Conferred by Vaccination

An LD50 titration is again conducted in immunocompetent B6 mice in a similar fashion as that described above, but utilizing animals previously immunized with the optimized dose of LAA polyprotein adsorbed to aluminum hydroxide (see example 5). Immunized animals are expected to survive a larger virus challenge (in terms of pfu per animal) than unimmunized B6 mice. An increase in LD50 value obtained from immunized B6 mice, as compared to unimmunized animals, would demonstrate the extent of protection obtained in vaccinated animals. An increase in LD50 value of at least one log (i.e., 10⁸ pfu in immunized animals compared to 10 pfu in unimmunized animals) is taken to indicate significant protection of immunized animals.

### Example 10

### Anti-LAA Antibodies Protect Against Vaccinia Infection

Cynomolgus monkeys are immunized IM with the optimal dosing schedule of polyprotein LAA (see example 5 and 7). Following the demonstration of the presence of antiviral antibodies in vaccinated animals by ELISA, vaccinated and control primates are injected intravenously with 10⁶ to 10⁹ pfu of variola major under anesthesia (LeDuc, J. W. et al. Emerging Inf. Dis. 8:743-745, 2002). The highest exposure is expected to be fatal within one week when administered to immunologically naive animals (Washington Times Jun. 2, 2002 available by hypertext transfer protocol://washingtontimes.com/national/20020602-2170892.htm). As smallpox is primarily spread by the inhalation of airborne particles, a second experiment utilizing an aerosol challenge can also be conducted.

The terms “a” and “an” and “the” and similar referents used in the context of describing the invention (especially in the context of the following claims) are to be construed to cover both the singular and the plural, unless otherwise indicated herein or clearly contradicted by context. Recitation of value ranges herein are merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. It is anticipated that one or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Of course, variations on those preferred embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference.

In closing, it is to be understood that the embodiments of the invention disclosed herein are illustrative of the principles of the present invention. Other modifications that can be employed are within the scope of the invention. Thus, by way of example, but not of limitation, alternative configurations of the present invention can be utilized in accordance with the teachings herein. Accordingly, the present invention is not limited to that precisely as shown and described

What is claimed is:

1. A polyprotein comprising external immunogens of membrane-associated proteins of variola major or immunologically cross-reactive poxviruses.

2. An immunogenic composition comprising the polyprotein of claim 1.

3. An isolated nucleic acid encoding the polyprotein of claim 1.

4. An immunogenic composition comprising the nucleic acid of claim 3.

5. A eukaryotic cell comprising the nucleic acid of claim 3.

6. The eukaryotic cell of claim 5, wherein the eukaryotic cell is a mammalian cell.

7. The polyprotein of claim 1 wherein the immunologically cross-reactive poxvirus is vaccinia virus.

8. A polyprotein comprising external immunogens of at least two poxvirus membrane-associated proteins selected from the group consisting of:
M1R, A36r, I5R, B7R, F8L, A30L, L1R, A33R, H5R, B5R, D8L and A27L.

9. The polyprotein of claim 8 comprising external immunogens of M1R, A30L, and A36R.

10. A polyprotein comprising external immunogens of at least two membrane-associated proteins, wherein antibodies against one of the proteins are synergistic with antibodies against the at least one other protein.

11. The polyprotein of claim 10 wherein the synergistic antibodies recognize A36R of variola major or A33R of vaccinia.

12. A method of inducing an antibody response comprising:
administering the polyprotein of claim 1 or 8 to a mammal.

13. A method of inducing an antibody response comprising:
administering the immunogenic composition of claim 2 or 4 to a mammal.

14. A method of making an immunogen comprising:
identifying a vaccinia protein that induces neutralizing or synergistic antibodies;
aligning the protein sequence of the vaccinia protein with its variola homolog;
synthesizing a nucleic acid sequence encoding at least an external segment of the variola protein; and
causing said nucleic acid to be expressed as a polypeptide.

15. The method of claim 14 wherein the causing step comprises transformation of a eukaryotic cell in vitro.

16. The method of claim 14 wherein the causing step comprises administration of the nucleic acid to a mammal.

17. A method of making an immunogen comprising:
identifying a vaccinia protein that induces neutralizing or synergistic antibodies;
aligning the protein sequences of multiple isolates of the vaccinia protein with multiple isolates its variola homolog;
determing a variola consensus sequence;
synthesizing a nucleic acid sequence encoding at least an external segment of said consensus sequence; and
causing said nucleic acid to be expressed as a polypeptide.

18. An immunogenic composition comprising an immunogen made according to claim 14 or 17.

19. An immunogenic composition comprising a cocktail of immunogens made according to claim 14 or 17.

20. An immunogenic composition comprising a complex of polypeptides wherein each polypeptide comprises an external immunogen of a membrane-associated protein of variola major or immunologically cross-reactive poxviruses.

21. The immunogenic composition of claim 18 wherein the polypeptides are biotinylated and the complex is formed by the addition of avidin or streptavidin.

22. The immunogenic composition of claim 18 wherein the complex is formed by anchoring the polypeptides in a liposome or micelle.

23. A polyprotein comprising external immunogens of membrane-associated proteins of variola major or immunologically cross-reactive poxviruses wherein the individual proteins are joined through a linker-spacer peptide.

24. The polyprotein of claim 23 wherein the linker-spacer peptide has the sequence GGGGSSGG.

25. The polyprotein of claim 23 further comprising an affinity tag.

26. The polyprotein of claim 25 wherein the affinity tag is a poly-histidine tag.


SEQUENCE LISTING (32 sequences)

SEQ ID NO: 1 - protein, 250 aa, Vaccinia virus
MGAAASIQTTVNTLSERISSKLEQEANASAQTKCDIEIGNFYIRQNHGCNLTVKNMCSADADAQLDAVLSAATETYSGLTPEQKAYVPAMFTAALNIQTSVNTVVRDFENYVKQTCNSSAVVDNKLKIQNVIIDECYGAPGSPTNLEFINTGSSKGNCAIKALMQLTTKATTQIAPKQVAGTGVQFYMIVIGVIILAALFMYYAKRMLFTSTNDKIKLILANKENVHWTTYMDTFFRTSPMVIATTDMQN

SEQ ID NO: 2 - protein, 250 aa, Vaccinia virus
MGAAASIQTTVNTLSERISSKLEQEANASAQTKCDIEIGNFYIRQNHGCNLTVKNMCSADADAQLDAVLSAATETYSGLTPEQKAYVPAMFTAALNIQTSVNTVVRDFENYVKQTCNSSAVVDNKLKIQNVIIDECYGAPGSPTNLEFINTGSSKGNCAIKALMQLTTKATTQIAPRQVAGTGVQFYMIVIGVIILAALFMYYAKRMLFTSTNDKIKLILANKENVHWTTYMDTFFRTSPMVIATTDMQN

SEQ ID NO: 3 - protein, 250 aa, Variola virus
MGAAASIQTTVNTLSERISSKLEQEANASAQTKCDIEIGNFYIRQNHGCNLTVKNMCSADADAQLDAVLSAATETYSGLTPEQKAYVPAMFTAALNIQTSVNTVVRDFENYVKQTCNSSAVVDNKLKIQNVIIDECYGAPGSPTNLEFINTGSSKGNCAIKALMQLTTKATTQIAPRQVAGTGVQFYMIVIGVIILAALFMYYAKRMLFTSTNDKIKLILANKENVHWTTYMDTFFRTSPMVIATTDIQN

SEQ ID NO: 4 - protein, 250 aa, Variola virus; identical to SEQ ID NO: 3

SEQ ID NO: 5 - protein, 250 aa, Artificial; identical to SEQ ID NO: 2
  Consensus sequence for SEQ ID NOs. 1-4

SEQ ID NO: 6 - protein, 110 aa, Vaccinia virus
MDGTLFPGDDDLAIPATEFFSTKAAKKPDRKREQIVKADEDDNEETLKQRLTNLEKKITNVTTKFEQIEKCCKRNDEVLFRLENHAETLRAAMISLAKKIDVQTGRRPYE

SEQ ID NO: 7 - protein, 110 aa, Vaccinia virus
MDGTLFPGDDDLAIPATEFFSTKADKKPEAKREAIVKADEDDNEETLKQRLTNLEKKITNVTTKFEQIEKCCKRNDEVLFRLENHAETLRAAMISLAKKIDVQTGRRPYE

SEQ ID NO: 8 - protein, 110 aa, Variola virus
MDGTLFPGDDDLAIPATEFFSTKAAKKPEAKREAIVKADGDDNEETLKQRLTNLEKKITNVTTKFEQIEKCCKRNDDVLFRLENHAETLRAAMISLAKKIDVQTGRRPYE

SEQ ID NO: 9 - protein, 110 aa, Variola virus; identical to SEQ ID NO: 8

SEQ ID NO: 10 - protein, 110 aa, Artificial; identical to SEQ ID NO: 8
  Consensus sequence for SEQ ID NOs. 6-9

SEQ ID NO: 11 - protein, 185 aa, Vaccinia virus
MMTPENDEEQTSVFSATVYGDKIQGKNKRKRVIGLCIRISMVISLLSMITMSAFLIVRLNQCMSANEAAITDAAVAVAAASSTHRKVASSTTQYDHKESCNGLYYQGSCYILHSDYQLFSDAKANCTAESSTLPNKSDVLITWLIDYVEDTWGSDGNPITKTTSDYQDSDVSQEVRKYFCVKTMN

SEQ ID NO: 12 - protein, 185 aa, Vaccinia virus; identical to SEQ ID NO: 11

SEQ ID NO: 13 - protein, 184 aa, Variola virus
MMTPENDEEQTSVFSATVYGDKIQGKNKRKRVIGICIRISMVISLLSMITMSAFLIVRLNQCMSANEAAITDATAVAAALSTHRKVASSTTQYKHQESCNGLYYQGSCYIFHSDYQLFSDAKANCATESSTLPNKSDVLTTWLIDYVEDTWGSDGNPITKTTTDYQDSDVSQEVRKYFCVKTMN

SEQ ID NO: 14 - protein, 184 aa, Variola virus; identical to SEQ ID NO: 13

SEQ ID NO: 15 - protein, 185 aa, Artificial
  Consensus sequence for SEQ ID NOs. 11-14.
MMTPENDEEQTSVFSATVYGDKIQGKNKRKRVIGICIRISMVISLLSMITMSAFLIVRLNQCMSANEAAITDAAVAVAAASSTHRKVASSTTQYKHQESCNGLYYQGSCYILHSDYQLFSDAKANCTAESSTLPNKSDVLTTWLIDYVEDTWGSDGNPITKTTSDYQDSDVSQEVRKYFCVKTMN

SEQ ID NO: 16 - protein, 304 aa, Vaccinia virus
MPQQLSPINIETKKAISNARLKPLDIHYNESKPTTIQNTGKLVRINFKGGYISGGFLPNEYVLSSLHIYWGKEDDYGSNHLIDVYKYSGEINLVHWNKKKYSSYEEAKKHDDGLIIISIFLQVLDHKNVYFQKIVNQLDSIRSANTSAPFDSVFYLDNLLPSKLDYFTYLGTTINHSADAVWIIFPTPINIHSDQLSKFRTLLSSSNHDGKPHYITENYRNPYKLNDDTQVYYSGEIIRAATTSPARENYFMRWLSDLRETCFSYYQKYIEENKTFAIIAIVFVFILTAILFFMSRRYSREKQN

SEQ ID NO: 17 - protein, 304 aa, Vaccinia virus
MPQQLSPINIETKKAISNARLKPLDIHYNESKPTTIQNTGKLVRINFKGGYISGGFLPNEYVLSSLHIYWGKEDDYGSNHLIDVYKYSGEINLVHWNKKKYSSYEEAKKHDDGLIIISIFLQVSDHKNVYFQKIVNQLDSIRSANTSAPFDSVFYLDNLLPSTLDYFTYLGTTINHSADAVWIIFPTPINIHSDQLSKFRTLLSSSNHDGKPYYITENYRNPYKLNDDTQVYYSGEIIRAATTSPARENYFMRWLSDLRETCFSYYQKYIEGNKTFAIIAIVFVFILTAILFLMSRRYSREKQN

SEQ ID NO: 18 - protein, 304 aa, Variola virus
MPQQLSPINIETKKAISNARLKPLNIHYNESKPTTIQNTGKLVRINFKGGYLSGGFLPNEYVLSSLHIYWGKEDDYGSNHLIDVYKYSGEINLVHWNKKKYSSYEEAKKHDDGLIIISIFLQVSDHKNVYFQKIVNQLDSIRTANTSAPFDSVFYLDNLLPSKLDYFKYLGTTINHSADAVWIIFPTPINIHSDQLSKFRTLLSLSNHEGKPHYITENYRNPYKLNDDTEVYYSGEIIRAATTSPARENYFMRWLSDLRETCFSYYQKYIEGNKTFAIIAIVFVYILTAILFLMSRRYSREKQN

SEQ ID NO: 19 - protein, 304 aa, Variola virus; identical to SEQ ID NO: 18

SEQ ID NO: 20 - protein, 304 aa, Artificial
  Consensus sequence for SEQ ID NOs. 16-19.
MPQQLSPINIETKKAISNARLKPLNIHYNESKPTTIQNTGKLVRINFKGGYISGGFLPNEYVLSSLHIYWGKEDDYGSNHLIDVYKYSGEINLVHWNKKKYSSYEEAKKHDDGLIIISIFLQVSDHKNVYFQKIVNQLDSIRSANTSAPFDSVFYLDNLLPSKLDYFTYLGTTINHSADAVWIIFPTPINIHSDQLSKFRTLLSSSNHDGKPHYITENYRNPYKLNDDTQVYYSGEIIRAATTSPARENYFMRWLSDLRETCFSYYQKYIEGNKTFAIIAIVFVFILTAILFLMSRRYSREKQN

SEQ ID NO: 21 - protein, 317 aa, Vaccinia virus
MKTISVVTLLCVLPAVVYSTCTVPTMNNAKLTSTETSFNDKQKVTFTCDQGYHSSDPNAVCETDKWKYENPCKKMCTVSDYISELYNKPLYEVNSTMTLSCNGETKYFRCEEKNGNTSWNDTVTCPNAECQPLQLEHGSCQPVKEKYSFGEYMTINCDVGYEVIGASYISCTANSWNVIPSCQQKCDMPSLSNGLISGSTFSIGGVIHLSCKSGFTLTGSPSSTCIDGKWNPVLPICVRTNEEFDPVDDGPDDETDLSKLSKDVVQYEQEIESLEATYHIIIVALTIMGVIFLISVIVLVCSCDKNNDQYKFHKLLP

SEQ ID NO: 22 - protein, 317 aa, Vaccinia virus
MKTISVVTLLCVLPAVVYSTCTVPTMNNAKLTSTETSFNNNQKVTFTCDQGYHSSDPNAVCETDKWKYENPCKKMCTVSDYISELYNKPLYEVNSTMTLSCNGETKYFRCEEKNGNTSWNDTVTCPNAECQPLQLEHGSCQPVKEKYSFGEYMTINCDVGYEVIGASYISCTANSWNVIPSCQQKCDIPSLSNGLISGSTFSIGGVIHLSCKSGFILTGSPSSTCIDGKWNPVLPICVRTNEEFDPVDDGPDDETDLSKLSKDVVQYEQEIESLEATYHIIIVALTIMGVIFLISVIVLVCSCDKNNDQYKFHKLLP

SEQ ID NO: 23 - protein, 317 aa, Variola virus
MKTISVVTLLCVLPAVVYSTCTVPTMNNAKLTSTETSFNDKQKVTFTCDSGYYSLDPNAVCETDKWKYENPCKKMCTVSDYVSELYNKPLYEVNAIITLICKDETKYFRCEEKNGNTSWNDTVTCPNAECQSLQLDHGSCQPVKEKYSFGEHITINCDVGYEVIGASYITCTANSWNVIPSCQQKCDIPSLSNGLISGSTFSIGGVIHLSCKSGFILTGSPSSTCIDGKWNPVLPICIRSNEEFDPVEDGPDDETDLSKLSKDVVQYEQEIESLEATYHIIIVALTIMGVIFLISVIVLVCSCNKNNDQYKFHKLLL

SEQ ID NO: 24 - protein, 317 aa, Variola virus; identical to SEQ ID NO: 23

SEQ ID NO: 25 - protein, 317 aa, Variola virus; identical to SEQ ID NO: 23

SEQ ID NO: 26 - protein, 317 aa, Variola virus; identical to SEQ ID NO: 23

SEQ ID NO: 27 - protein, 317 aa, Artificial; identical to SEQ ID NO: 23
  Consensus sequence for SEQ ID NOs. 21-26.

SEQ ID NO: 28 - protein, 203 aa, Vaccinia virus
MAWSITNKADTSSFTKMAEIRAHLKNSAENKDKNEDIFPEDVIIPSTKPKTKRATTPRKPAATKRSTKKEEVEEEVVIEEYHQTTEKNSPSPGVSDIVESVAAVELDDSDGDDEPMVQVEAGKVNHSARSDLSDLKVATDNIVKSLKKIITRISAVSTVLEDVQAAGISRQFTSMTKAITTLSDLVTEGKSKVVRKKVKTCKK

SEQ ID NO: 29 - protein, 203 aa, Vaccinia virus
MAWSITNKADTSSFTKMAEIRAHLKNSAENKDKNEDIFPEDVIIPSTKPKTKRATTPRKPAATKRSTKKEEVEEEVVIEEYHQTTEKNSPSPGVGDIVESVAAVELDDSDGDDEPMVQVEAGKVNHSARSDLSDLKVATDNIVKSLKKIITRISAVSTVLEDVQAAGISRQFTSMTKAITTLSDLVTEGKSKVVRKKVKTCKK

SEQ ID NO: 30 - protein, 221 aa, Variola virus
MAWSITNKADTSSFTKMAEIRAHLRNSAENKDKNDDIFPEDVIIPSTKPKTKRATTPRKPAATKRSTKKDKEKEEVEEEEVVIEEYHQTTEENSPPPSSSPGVGNIVESVTAVELDDSNGDDDNDNDNDDNEPMVQVEAGKVNHSARSDLSDLKVATDNIVKSLKKIITRISAVSTVLEDVQAAGISRQFTSMTKSITTLSDLVTEGKSKVVRKKVKTCKK

SEQ ID NO: 31 - protein, 220 aa, Variola virus
MAWSITNKADTSSFTKMAEIRAHLRNSAENKDKNDDIFPEDVIIPSTKPKTKRATTPRKPAATKRSTKKDKEKEEVEEEVVIEEYHQTTEENSPPPSSSPGVGDIVESVTAVELDDSNGDDDNDNDNDDNEPMVQVEAGKVNHSARSDLSDLKVATDNIVKSLKKIITRISAVSTVLEDVQAAGISRQFTSMTKSITTLSDLVTEGKSKVVRKKVKTCKK

SEQ ID NO: 32 - protein, 221 aa, Artificial
  Consensus sequence for SEQ ID NOs. 28-31.
MAWSITNKADTSSFTKMAEIRAHLKNSAENKDKNDDIFPEDVIIPSTKPKTKRATTPRKPAATKRSTKKDKEKEEVVEEEVVIEEYHQTTEKNSPPPSSSPGVGDIVESVTAVELDDSNGDDDNDNDNDDNEPMVQVEAGKVNHSARSDLSDLKVATDNIVKSLKKIITRISAVSTVLEDVQAAGISRQFTSMTKAITTLSDLVTEGKSKVVRKKVKTCKK